(19) United States
(12) Patent Application Publication
(10) Pub. No.: US 2007/0072917 A1
(43) Pub. Date: Mar. 29, 2007

(54) Substituted 2-aminotetralin for the treatment of depression

(75) Inventors: Dieter Scheller, Neuss (DE); Alexander Breidenbach, Weil am Rhein (DE); Norma Selve, Troisdorf (DE)

Correspondence Address: HARNESS, DICKEY, & PIERCE, P.L.C, 7700 BONHOMME, STE 400, ST. LOUIS, MO, 63105, US

(73) Assignee: SRZ Properties, Inc., Wilmington (DE)

(21) Appl. No.: 10/565,713

(22) PCT Filed: Jul. 22, 2004
(86) PCT No.: PCT/EP04/08169

§ 371 Date: Jan. 25, 2006

(30) Foreign Application Priority Data
Jul. 26, 2003 (DE) 103 34 187.0

(51) Int. Cl. A61K 31/44; A61K 31/4172; A61K 31/405; A61K 31/40; A61K 31/381; A61K 31/343; A61K 31/195; A61K 31/165; A61K 31/137

(52) U.S. Cl. 514/357; 514/396; 514/415; 514/443; 514/408; 514/438; 514/471; 514/567; 514/621; 514/649

(57) ABSTRACT

The invention relates to the use of a compound of general formula (I) and the pharmaceutically acceptable salts, racemates or pure enantiomers thereof for the production of a medicament used to treat depression. The substituents are defined as in the description.

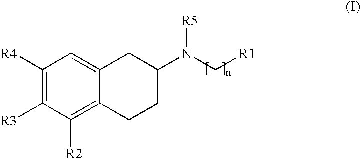

According to estimates by the WHO, depression will be the second most common cause of illness-related disability by the year 2020 (Murray, Lancet 349 (1997) 1498). The efficiency of current pharmacological treatments is limited for various reasons, for example owing to a late onset of effect, side effects or a lack of effectiveness of the medicament. Owing to the frequency and duration of this illness and to the tendency to relapse, there is a great need for new, innovative antidepressants.

Substituted 2-aminotetralins are known from U.S. Pat. No. 4,564,628, U.S. Pat. No. 4,885,308, U.S. Pat. No. 4,722,933 and WO 01/38321. These are substances with a dopaminergic effect, which are known in particular for the treatment of Parkinson's disease. In clinical studies, the rotigotine [(−)-5,6,7,8-tetrahydro-6-[propyl[2-(2-thienyl)ethyl]amino]-1-naphthol] in particular proved to be an effective, transdermally available anti-Parkinson drug (Metman, Clinical Neuropharmacol. 24, 2001, 163).

It has now been surprisingly found that said substituted 2-aminotetralins of the general formula I 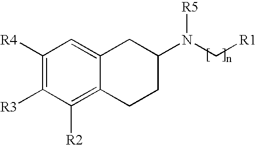
wherein:
n is 1-5;
R2 is OA; R3 and R4 are each independently selected from H and OA; with A being selected from H, C1-3 alkyl or a group 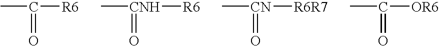
wherein R6 and R7 are each independently alkyl, in particular C1-20 alkyl, or aryl, in particular optionally substituted phenyl;
R5 is a C1-3 alkyl;
R1 is a group selected from hydrogen, 3-pyridyl, 4-pyridyl, optionally substituted phenyl, 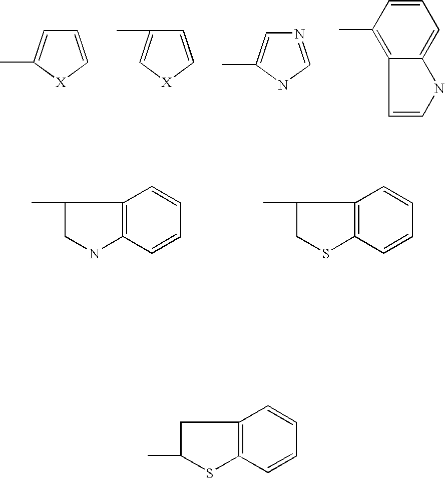
wherein X is selected from S, O or NH;
wherein the compound of formula I can be present as racemate or as a pure (R)- or (S)-enantiomer,
as well as physiologically acceptable salts of these compounds are suitable for the production of medicaments for the treatment of depression.

Compounds that are particularly suitable for the production of an antidepressant are those in which R2 is an OA group and R3 and R4 are independently H or an OA group, it being particularly preferred for A to be selected from a hydrogen atom or a group 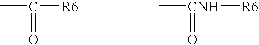
in which R6 is a C1-20 alkyl, in particular C1-12 alkyl, phenyl or methoxyphenyl.

In another preferred embodiment of the invention R4 is H.

In another preferred embodiment of the invention R3 is H.

In another preferred embodiment of the invention R3 and R4 are both H.

In another preferred embodiment of the invention n=1, 2 or 3.

In another preferred embodiment of the invention R3 and R4 are both H and R2 is —OH or —O(CO)CH3, it being especially preferred for n to be 2.

R1 is preferably selected from the group 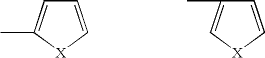
wherein X is selected from S, O and NH and wherein it is especially preferred for X to be a sulphur atom.

It is especially preferred for R1 to be 2-thienyl.

In a further preferred embodiment of the invention, R5 is a C3-alkyl.

In a particularly preferred embodiment of the invention, the racemate of (+/−) 5,6,7,8-tetrahydro-6-[propyl[2-(2-thienyl)ethyl]amino]-1-naphthol, and especially preferred the pure S-enantiomer of this compound (rotigotine), is used for the production of the medicament for the treatment of depression.

The expressions “C1-20 alkyl”, “C1-12 alkyl” and “C1-3 alkyl” are each to be understood as branched or non-branched alkyl groups with the corresponding number of C atoms. For example, a “C1-20 alkyl” includes all alkyls with 1 to 20 C atoms. The alkyls can be optionally substituted, e.g. with halogen. The alkyls are preferably present in non-substituted form.

The suitability of rotigotine as an antidepressant was demonstrated in three different, validated animal models.

The “forced swim test” is an animal model in which depressive episodes are triggered by acute stress. In this test, rats are forced to swim in a limited space. After initial attempts to save themselves, in which the animals realise the hopelessness of the situation, they lapse into immobility. On repetition of the experiment, the animals remain immobile from the start of the experiment. If the animals are pre-treated with antidepressants, the period of immobility in the repeated test is shortened and the animals generally commence search and escape movements immediately after transfer into the water basin (Porsolt, Biomedicine 30, 1979, 139). Rotigotine leads to a significantly shortened period of immobility.

In the “learned helplessness test”, rats are repeatedly subjected to uncontrollable stress. This brings about an impaired learning ability in the animals in a later situation (for example after 48 hours) in which they could escape the stress again. Following the sub-chronic but not acute administration of antidepressants, the learning ability normalises again and the animals learn to escape the (announced) stress (in time) (Sherman, Pharmacology Biochemistry & Behavior 16, 1982, 449). After several days of administration of rotigotine depot suspensions (embodiment 2), the animals exhibited an improved learning behaviour at low concentrations; however the higher doses also increased the activity of the animals under non-test conditions.

In a further animal model (embodiment 3), it was examined whether the antidepressive effects of rotigotine can be distinguished from a general motor stimulation. In this case, rotigotine was administered to rats whose olfactory bulbs had been removed on both sides. The removal of the olfactory bulb leads to an adaptive hyperactivity in the untreated control group. It is known from literature that chronically administered antidepressants lead to a reduction in the movement activity of the animals in this model, whereas stimulants further increase the motor activity (van Riezen H et al, Br J. Pharmacol. 60(4), 1977, 521; Kelly J P et al, Pharmacol Ther. 74(3), 1997, 299). Therefore, it is possible to discriminate between antidepressive and non-specific stimulatory effects of an active ingredient with this model. It has been shown that rotigotine exhibits a specifically antidepressive effect in low doses, which approximately corresponds to the effect of the antidepressant imipramine and which leads to almost complete suppression of the bulbectomy-induced locomotor hyperactivity. In the case of higher rotigotine concentrations on the other hand, the stimulatory dopamine-agonistic effect is dominant.

It could thus be clearly shown that subcutaneously applied rotigotine surprisingly had a significant antidepressive effect in all three tests.

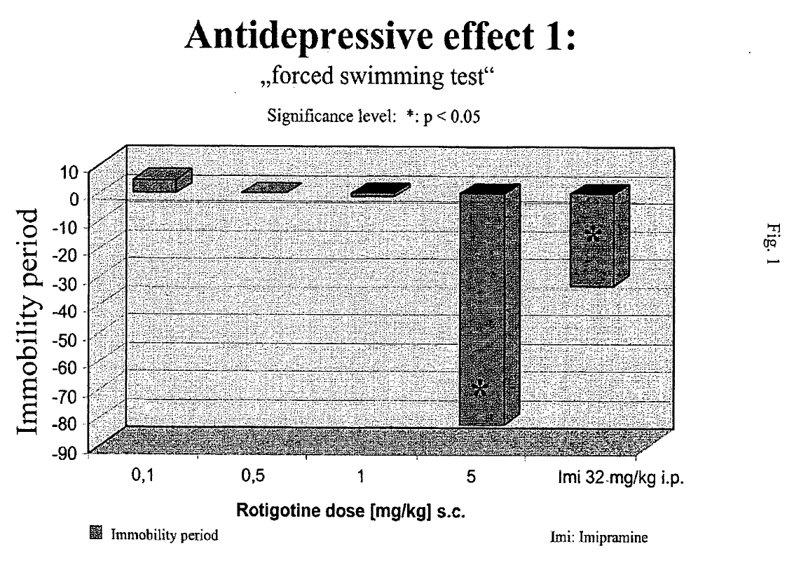

FIG. 1 shows that rotigotine leads to a clear reduction in the period of immobility in the “forced swim test”.

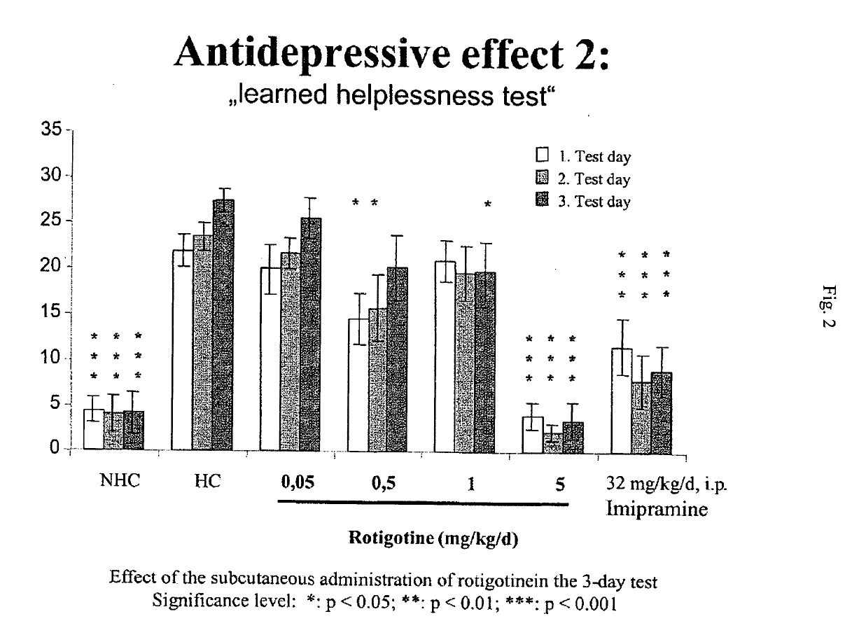

FIG. 2 shows that animals treated with rotigotine depot suspension (embodiment 2) in the “learned helplessness test” exhibit, depending on the dose, a normalised learning behaviour (NHC) as compared to the control group (HC) treated only with vehicle.

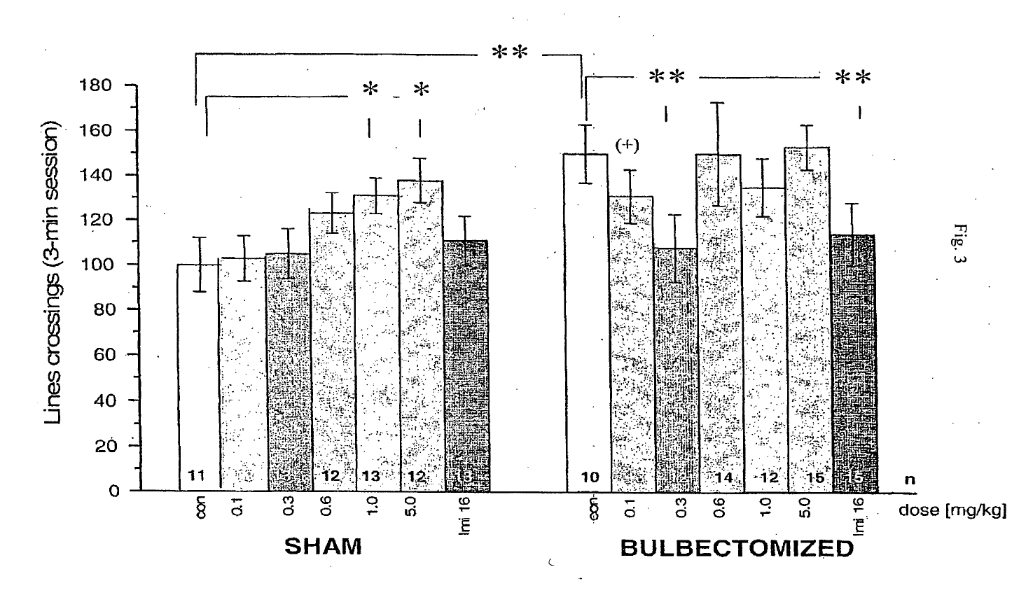

FIG. 3 shows that in bulbectomised rats (embodiment 3), low doses of rotigotine significantly reduce motor hyperactivity and thus a clear antidepressive effect develops. In higher doses on the other hand, a non-specific activation of the locomotor activity dominates and occurs in both bulbectomised animals as well as in control animals.

It can be concluded from the preclincal data that new effective medicaments for the treatment of depression can be made available with the substituted 2-aminotetralins of the general formula I which are known as anti-Parkinson drugs.

A subject matter of the invention is therefore the use of compounds of formula I, in particular rotigotine, as well as salts of these compounds for the production of a medicament for the treatment of depression.

In this patent application, the term “treatment” includes both the therapy of existing depression and also the preventative therapy (prophylaxis) of depression, for example of recurrent depressive phases.

For a better understanding and in order to achieve an optimum individual therapy, depressive disorders are divided into sub-forms, whereby the transitions between the various sub-forms are often blurred. Depression is classified—traditionally—according to its presumed causes or—more recently—according to its symptoms (see in this regard ICD-10 “International Statistical Classification of Diseases and Related Health Problems” of the WHO).

In this patent application, the term “depression” is taken to mean both the various traditional sub-forms of depression as cited below as well as the disorders subsumed under the term “affective disorders” in the ICD-10, which accompany depressive episodes, in particular depressive episodes, recurrent depressive disorders, depressive phases in bipolar affective disorders as well as anxiety disorders, adjustment disorders and organic brain diseases which are each accompanied by depressive symptoms. Corresponding disorders are listed, for example, in the ICD-10 classifications (version 2.0, November 2000) F31, F32, F33, F41, F43, F45 and F06.

If depression is classified in the traditional manner according to cause, 4 main classes are generally discerned:

I. Endogenous Depression

In the case of endogenous depression, no easily discernable external causes can be identified as triggers of the depression. Triggers are probably disorders of the neurotransmitter system of the brain. The phase-like course wherein the depressive episodes can repeatedly occur is typical of endogenous depression. Endogenous depression is generally subdivided into
   unipolar depression (“major depression”), in which only depressive phases occur,
   bipolar depression (“manic-depressive disorders”), in which depressive episodes alternate with manic phases.
      II. Somatogenic Depression
Physical-organic disorders are the cause of this depression. Somatogenic depression is generally subdivided into
   organic depression which is based on an illness or injury to the brain. Such illnesses or injuries, which are often accompanied by a changed brain metabolism, are, for example, brain tumours, Parkinson's disease, migraines, epilepsy, brain paralysis, arteriosclerosis of the brain, brain traumas, meningitis, strokes and dementias, such as, for example, Alzheimer's disease;
   symptomatic depression, which often occurs as a result of or as an accompaniment to an illness which only indirectly influences the brain function. This may be, for example, a circulatory illness, hypothyroidism or another hormone disorder, an infectious disease, cancer or liver disease;
   pharmacogenic depression, for example in the case of alcohol, medication or drug misuse.
      III. Psychogenic Depression
This depression is often an overreaction to one or more traumatic experiences. It is frequently subdivided into exhaustion depression, neurotic depression and reactive depression as a result of current conflicts or events.
   IV. Depression in Specific Life Situations
Examples are postpartum depression, old-age depression, childhood depression, seasonal depression as well as pubertal depression.

Compounds of formula I, in particular rotigotine, as well as the salts thereof are basically suitable for the production of a medicament for the treatment of the various forms of depression mentioned above or for the treatment of affective disorders, in particular depressive episodes, recurrent depressive disorders, cyclothymia and depressive phases in bipolar affective disorders, according to ICD-10.

According to the invention, compounds of formula I are preferably used for the production of a medicament for the treatment of depressive episodes and serious recurrent depressive disorders such as those occurring, for example, in the case of endogenous, unipolar depression (“major depression”).

Metabolic disturbances of the brain cells, i.e. a lack of noradrenaline or serotonin, and/or a genetic predisposition are regarded as causes of endogenous, unipolar depression.

In this patent application, the expression “major depression” includes a disorder as described in the American diagnosis manual “The Diagnostic and Statistic Manual of Mental Disorders—4th Edition” (American Psychiatric Association, 1994; “DSM IV”).

The compounds of formula I, in particular rotigotine, and the salts thereof are also particularly suitable for the production of antidepressants for the treatment of depressive episodes in manic-depressive patients. In this patent application, these depressive phases in bipolar disorders are subsumed under the term “depression”.

Furthermore, the compounds of formula I are preferably used for the production of a medicament for the treatment of “organic” depression which is described above. Organic depression often occurs, for example, in Parkinson's disease or in cerebrovascular diseases and in dementia disorders.

When treating depressions occurring as a consequence of Parkinson's disease, the conclusion which is relevant for clinical practice can be drawn from the present invention that the conventional co-medication of antidepressants and anti-Parkinson drugs is not required if the depressive Parkinson's patients are put on compounds of formula I, in particular rotigotine.

A subject matter of the invention is therefore the use of compounds of formula I, in particular rotigotine, and salts of these compounds for the production of a medicament for the treatment of depression linked with Parkinson's disease, whereby co-medication with other antidepressants can be optionally forgone.

Another subject matter of the invention is the use of compounds of formula I, in particular rotigotine, as well as salts of these compounds, in each case alone or in combination with other antidepressants, for the treatment of organic depression which is not linked with Parkinson's disease. Examples of such organic depression include depression associated with brain tumours, migraines, epilepsy, brain paralysis, brain arteriosclerosis, brain traumas, meningitis, strokes, dementia, Alzheimer's disease or Parkinson Plus Syndrome.

A further subject matter of the invention is a method for the treatment of depression in a mammal, in particular endogenous, unipolar depression (“major depression”), a depressive phase of a bipolar disorder, Parkinson's-related depression or an organic depression which is not associated with Parkinson's disease, by administering a therapeutically effective quantity of one of the compounds of formula I, in particular rotigotine, as well as salts of these compounds to said mammal, in particular to a human.

Compounds of formula I are optically active and can be present as racemates or as pure (R)- or (S)-enantiomers. The expression “pure enantiomer” is understood in this patent application to mean that a substance is preferably present at least 90 mol %, particularly preferred at least 95, 98 or 99 mol %, in the form of one enantiomer, e.g. in the (S) form, whereas the proportion of the respective other enantiomer, e.g. the (R) form, is correspondingly low. If, for example, rotigotine [(−)-5,6,7,8-tetrahydro-6-[propyl[2-(2-thienyl)ethyl]amino]-1-naphthol] is used to produce the medicament according to the invention, the (R)-(+)-enantiomer is preferably present with a proportion of <10 mol %, particularly preferred with a proportion of <2 mol % and especially preferred with a mole proportion of <1%, based on the total amount of rotigotine in the antidepressant.

Compounds of formula I can be present in the medicament as free bases or in the form of the physiologically acceptable salts, e.g. in the form of rotigotine hydrochloride.

“Physiologically acceptable salts” include non-toxic addition salts of a base, in particular a compound of formula I in the form of the free base, with organic or inorganic acids, such as, for example, HCl.

There are many methods of application available for administering compounds of formula I, which the person skilled in the art can select and adapt depending on the need, condition and age of the patient, the required dosage and the desired application interval.

A preferred mode of administering compounds of formula I is transdermal administration. The form of administration may, in principle, be selected from, for example, an ointment, a paste, a spray, a film, a plaster (patch) or an iontophoretic device.

Compounds of formula I, for example rotigotine, are preferably applied in plaster form to the skin of the patient, wherein the active ingredient is preferably present in a matrix of adhesive polymer, for instance a self-adhesive polysiloxane (embodiment 1). Examples of suitable transdermal formulations can be found in WO 99/49852, WO 02/89777 and WO 02/89778. Such a form of administration enables a substantially constant plasma level to be established and therefore a constant dopaminergic stimulation over the entire application interval (WO 02/89778; Metman, Clinical Neuropharmacol. 24, 2001, 163).

If, on the other hand, an antidepressant in the form of a subcutaneous or intramuscular depot form is desired, a compound of formula I may be suspended, for example as a salt crystal, for instance as a crystalline hydrochloride, in a hydrophobic anhydrous medium and injected, such as described in WO 02/15903, or else administered in the form of microcapsules, microparticles or implants based on biodegradable polymers, such as described, for example, in WO 02/38646.

Other conceivable forms of administering compounds of formula I are transmucosal formulations, for example sublingual sprays, rectal formulations or aerosols for pulmonary administration.

Suitable dosages of compounds of formula I are generally between 0.1 and approximately 50 mg/day, with daily doses of preferably between 0.2 and 40 mg and in particular of between 0.4 and 20 mg/day being administered. Particularly preferred dosages of compounds of formula I, in particular rotigotine, are greater than 0.5 mg/day, whereby for applications that do not require the simultaneous treatment of Parkinson's disease motor disorders, it is especially preferred for dosage forms to be selected in which the antidepressive effect of compounds of formula I, in particular of rotigotine, is pronounced, but in which the non-specific stimulatory effect of compounds of formula I, in particular of rotigotine, is as low as possible. Such dosages are generally less than 10 mg/day, for example less than 7.5 mg or less than 5, 4, 3, 2 or less than 1 mg/day, and in particular between 0.5 and 5 mg/day.

However, in patients with Parkinson's disease, a dosage of sometimes greater than 5 mg/day may be required for the simultaneous therapy of the motor disorders. Depending, for example, on the age and condition of the patient, the degree of severity of the illness etc, corresponding dosages are sometimes significantly greater than 1 mg/day, for example greater than 5, 6, 8, 9, 10 or even between 10 and 50 mg/day, for example between 10 and 25 mg/day.

The desired daily dose may be controlled by the design of the formulation depending on the type of application selected. For example, the daily dose of transdermally administered compounds of formula I, in particular rotigotine, can be adjusted by adjusting a corresponding flux rate per unit of area and/or by varying the size of the plaster. Dosage can thereby take place in a gradually increasing manner, i.e. the treatment may optionally start with low dosages which are then increased to the maintenance dose.

A subject matter of the invention is therefore a dosage form, for example a plaster or an injectable depot formulation, which releases the appropriate amount of the compound of formula I required for therapy of the depression, for example between 0.5 and 10 mg/day or between 0.5 and 5 mg/day, as described above.

It is clear to the person skilled in the art that the dosage interval may vary depending on the applied quantity, the mode of application and the daily requirement of the patient. Thus, a transdermal form of application may be designed, for example, for administration once a day, once every three days or once every seven days, whilst a subcutaneous or intramuscular depot can make it possible to administer injections, for example, in one-weekly, two-weekly or four-weekly cycles.

Compounds of formula I, in particular rotigotine, can be used for the monotherapy of depression. However, in one embodiment of the invention, other active ingredients in addition to compounds of formula I may also be present in the antidepressive medicament form.

Examples hereof are other antidepressants which directly or indirectly influence the serotonin or noradrenaline metabolism.

Examples hereof are
selective serotonin reuptake inhibitors, such as sertraline, citalopram, paroxetine or fluoxetine
mixed serotonin-, noradrenaline reuptake inhibitors such as venlaxafine, milnacipram, mirtazapine and tricyclic antidepressants such as amitryptiline and imipramine
selective noradrenaline reuptake inhibitors such as reboxetine
monoaminoxidase inhibitors such as tranylcypramine or clorgyline
alpha2-receptors and/or serotonin receptor-modulators such as mirtazapine or nefazodone.

Other examples of antidepressants are adenosine antagonists, such as for example, ST 1535, sigma-opioid receptor ligands, NK antagonists such as GW 597599, saredudant or aprepitant, melatonin agonists or modulators of the hypothalamus-hypophysis-adrenal axis.

Depending on the cause and the symptoms of the depression, a combination preparation may also contain an additional antipsychotic, sedative, anxiolytic or anti-migraine agent, or an active ingredient which displays one or more effects selected from an antidepressive, antipsychotic, sedative, anxiolytic or anti-migraine effect.

The compound of formula I and the additional antidepressant, antipsychotic, sedative, anxiolytic or anti-migraine agent may thereby be present in the same pharmaceutical formulation, for example in a combination tablet, or also in different application units, for example in the form of two separate tablets. The two active ingredients may be administered simultaneously or at separate times as required.

In a combination preparation, a sequential administration can be achieved, for example, in that an administration form, for example an oral tablet, has two different layers with differing release profiles for the different pharmaceutically active ingredients. It is clear to the person skilled in the art that various forms of administration and application patterns are conceivable within the context of the present invention, which all form subject matter of the invention.

Examples of antipsychotics are promethazine, fluphenazine, perphenacine, levomepromazine, thioridazine, perazine, promazine, chlorprothixene, zuclopenthixol, prothipendyl, flupentixol, zotepine, benperidol, pipamperone, melperone, haloperidol, bromperidol, sulpiride, clozapine, pimozide, risperidone, quetiapine, amisulpride, olanzapine.

Examples of sedatives are diphenhydramine, doxylamine succinate, nitrazepam, midazolam, lormetazepam, flunitrazepam, flurazepam, oxazepam, bromazepam, triazolam, brotizolam, temazepam, chloral hydrate, zopiclone, zolpidem, tryptophan, zaleplon.

Examples of anxiolytics are fluspirilene, thioridazine, oxazepam, alprazolam, bromazepam, lorazepam, prazepam, diazepam, clobazam, medazepam, chlordiazepoxide, dipotassium clorazepate, nordazepam, meprobamate, buspirone, kavain, hydroxyzine.

Examples of anti-migraine agents are almotriptan, zolmitriptan, acetylsalicylic acid, ergotamine, dihydroergotamine, methysergide, iprazochrome, ibuprofen, sumatriptan, rizatriptan, naratriptan, paracetamol.

### EMBODIMENTS

### Embodiment 1

### Rotigotine Plaster

1.8 g of rotigotine (free base) are dissolved in 2.4 g of ethanol and added to 0.4 g of Kollidon 90F (dissolved in 1 g of ethanol). This mixture is added to a 74% solution of silicone polymers (8.9 g of BioPSA 7-4201+8.9 g of BIO-PSA 7-4301 [Dow Corning]) in heptane. Following the addition of 2.65 g of petrol ether, the mixture was stirred for 1 hour at 700 rpm in order to obtain a homogeneous dispersion. Following lamination on polyester, it was dried at 50° C. The final weight of the plaster was 50 g/cm².

### Embodiment 2

### Rotigotine Depot Suspensions

(a) 1411.2 g of Miglyol 812 were weighed into a Duran flask. 14.4 g of Imwitor 312 were added to the Miglyol and then heated for 30 minutes to 80° C. whilst being stirred. The clear solution was cooled to room temperature and filtered.

(b) 1188 g of the solution produced in (a) were transferred into a glass laboratory reactor, 12 g of rotigotine were added and homogenised for 10 minutes under nitrogen with an Ultraturrax at 10,000 rpm. The suspension was decanted into brown glass bottles whilst the Ultraturrax was running (2,000 rpm).

### Embodiment 3

The bulbectomy study was carried out on Sprague-Dawley rats. A sham-operated group, which was operated on without removal of the olfactory bulbs, served as a control group. 14 days after the operation, the rats were treated with vehicle, a rotigotine depot suspension (every second day) or imipramine. On test days, the rats were placed onto a test field and left to themselves for 3 minutes. The locomotor activities of the animals were thereby measured on the basis of the number of lines crossed.

1-15. (canceled)

16. A combination preparation comprising (a) a compound having the formula 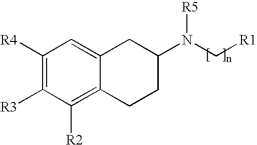
wherein
n is a number from 1 to 5;
R2 is OA, and R3 and R4 are each independently selected from H and OA, where A is H, C₁₋₃ alkyl or a group 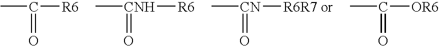
where R6 and R7 are each independently alkyl or aryl;
R5 is C₁₋₃ alkyl;
R1 is a group 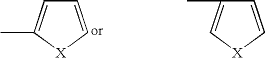
where X is S, O or NH;
or a racemate or pure (R)- or (S)-enantiomer thereof, or a physiologically acceptable salt thereof; and (b) at least one further active ingredient selected from the group consisting of antidepressants, antipsychotics, sedatives, anxiolytics and anti-migraine agents.

17. A method for treating depression in a mammal, comprising administering to the mammal a therapeutically effective amount of a compound having the formula 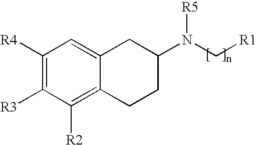
wherein
n is a number from 1 to 5;
R2 is OA, and R3 and R4 are each independently selected from H and OA, where A is H, C₁₋₃ alkyl or a group 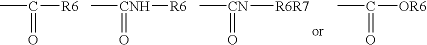
where R6 and R7 are each independently alkyl or aryl;
R5 is C₁₋₃ alkyl;
R1 is a group 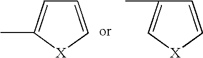
where X is S, O or NH;
or a racemate or pure (R)- or (S)-enantiomer thereof or a physiologically acceptable salt thereof.

18. The method of claim 17, wherein, in the formula for said compound, R3 and R4 are both H.

19. The method of claim 17, wherein, in the formula for said compound, A is H or a group 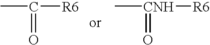
where R6 is C₁₋₁₂ alkyl, phenyl or methoxyphenyl.

20. The method of claim 17, wherein, in the formula for said compound, n is a number from 1 to 3 and R5 is C₃ alkyl.

21. The method of claim 17, wherein, in the formula for said compound, X is S.

22. The method of claim 21, wherein, in the formula for said compound, R1 is a 2-thienyl group.

23. The method of claim 17, wherein the compound is 5,6,7,8-tetrahydro-6-[propyl-[2-(2-thienyl)ethyl]amino]-1-naphthol.

24. The method of claim 17, wherein the mammal is human.

25. The method of claim 24, wherein the depression is an endogenous depression or an organic depression not associated with Parkinson's disease.

26. The method of claim 24, wherein the depression is a unipolar depression (major depression) or a depressive phase of a manic-depressive disorder.

27. The method of claim 24, wherein the depression is an organic depression not associated with Parkinson's disease.

28. The method of claim 24, wherein the depression is an organic depression associated with Parkinson's disease.

29. The method of claim 28, wherein co-medication with another antidepressant is absent.

30. The method of claim 24, wherein the compound, or racemate or enantiomer thereof, or salt thereof, is administered parenterally, transdermally or mucosally.

31. The method of claim 24, wherein the compound, or racemate or enantiomer thereof, or salt thereof, is formulated as an ointment, paste, spray, film, plaster or iontophoretic device for transdermal administration.

32. The method of claim 24, wherein the active ingredient is administered transdermally via a plaster having the active ingredient in a matrix comprising an adhesive polymer.

33. The method of claim 24, wherein the active ingredient is administered transdermally and wherein a substantially constant plasma level of the active ingredient is established.

34. The method of claim 24, wherein the compound, or racemate or enantiomer thereof, or salt thereof, is administered in a dose of 0.5 to 50 mg per day.

35. The method of claim 17, further comprising administering to the mammal an additional active ingredient selected from the group consisting of antidepressants, antipsychotics, sedatives, anxiolytics and anti-migraine agents.

36. The combination preparation of claim 16, wherein the additional active ingredient is an antidepressant selected from the group consisting of selective serotonin reuptake inhibitors, mixed serotonin and noradrenaline reuptake inhibitors, selective noradrenaline reuptake inhibitors, monoamine oxidase inhibitors, alpha2 receptor and/or serotonin receptor modulators, adenosine antagonists, sigma-opioid receptor ligands, NK antagonists, melatonin antagonists and modulators of the hypothalamus-hypophysis-adrenal axis.
